(19) United States
(12) Patent Application Publication
(10) Pub. No.: US 2016/0310704 A1
(43) Pub. Date: Oct. 27, 2016

(54) NEEDLE ASSEMBLY WITH SEALED NOTCH AND RELATED METHODS

(71) Applicant: B. BRAUN MELSUNGEN AG, Melsungen (DE)

(72) Inventors: Kooi Sim Ng, Gelugor (MY); Siew Ping Lim, Georgetown (MY); Riduan Bin Tajudin, Bayan Lepas (MY); Beng Hor Sum, Jelutong (MY); Mohd Zairizal Bin Zakaria, Bayan Lepas (MY)

(73) Assignee: B. Braun Melsungen AG, Melsungen (DE)

(21) Appl. No.: 15/100,894

(22) PCT Filed: Dec. 3, 2014
(86) PCT No.: PCT/EP2014/076461

§ 371 Date: Jun. 1, 2016

Related U.S. Application Data
(60) Provisional application No. 61/911,887, filed on Dec. 4, 2013.

(51) Int. Cl. A61M 25/06; A61M 5/32; A61M 25/00

(52) U.S. Cl. CPC A61M 25/0606; A61M 25/0009; A61M 25/0618; A61M 5/329; A61M 25/0631; A61M 2205/583; A61M 25/0693

(57) ABSTRACT

Needle devices are disclosed in which a needle having a wall defining a needle lumen has a notch (120) formed through the wall. A transparent or semi-opaque window cover (130) made from a biocompatible material is provided over the notch. Blood flow through the needle lumen can be viewed through the notch with the window cover. The needle with the notch and window cover may be used with a variety of needle applications, including as catheter assemblies, indwelling assemblies, and most if not all over-the-needle type devices.

### CROSS-REFERENCE TO RELATED APPLICATIONS

This application is the national phase entry, under 35 U.S.C. Section 371(c), of International Application No. PCT/EP2014/076461, filed Dec. 3, 2014, claiming priority from U.S. Patent Application No. 61/911,887, filed Dec. 4, 2013. The disclosures of the International Application and the U.S. Application from which this application claims priority are incorporated herein by reference in their entireties.

### FIELD OF ART

This invention relates generally to needle devices, systems, and methods for use where medicines are delivered vascularly. More specifically, the present disclosure relates to catheter devices and needle configurations used in intravenous medical devices and methods for using and making such devices and systems.

### BACKGROUND

Generally, vascular access devices are used for communicating fluid with the vascular system of patients. For example, catheters are used for infusing fluid, such as normal saline solution, various medicaments, and total parenteral nutrition, into a patient, withdrawing blood from a patient, or monitoring various parameters of the patient's vascular system.

A common type of intravenous (IV) catheter is an over-the-needle peripheral IV catheter. As its name implies, an over-the-needle catheter is mounted over an introducer needle having a sharp distal tip. At least the inner surface of the distal portion of the catheter tightly engages the outer surface of the needle to prevent peelback of the catheter and thus facilitate insertion of the catheter into the blood vessel. The catheter and the introducer needle are assembled so that the distal tip of the introducer needle extends beyond the distal tip of the catheter with the bevel of the needle facing up away from the patient's skin. The catheter and introducer needle are generally inserted at a shallow angle through the patient's skin into a blood vessel.

In order to verify proper placement of the needle and catheter in the blood vessel, the clinician generally looks for blood flashback as confirmation of the access. The first blood flashback is through the needle and into a transparent needle hub, which is sometimes referred to as primary blood flashback. This confirms at least the needle has found the vein. Then as the needle is withdrawn in a proximal direction away from the catheter tube and the blood will flash back between the needle and the catheter. This is sometimes referred to as a secondary flashback, which confirms that the catheter tube has found the vein. Once proper placement of the catheter into the blood vessel is confirmed, the clinician may apply pressure to the blood vessel by pressing down on the patient's skin over the blood vessel distal of the introducer needle and the catheter. This finger pressure occludes the vessel, minimizing further blood flow through the introducer needle and the catheter.

In some IV catheter assemblies, the needle has an open notch, through which blood can flow into the space between the needle and catheter. This “instant flash” confirms only that the needle tip has entered the vein but not the necessarily the catheter tube has entered the vein. Because there is first blood between the needle and the catheter tube when a notch is employed, a secondary flashback is not possible.

The clinician may then withdraw the introducer needle from the catheter. The introducer needle may be withdrawn into a needle tip shield or needle cap that covers the needle tip and prevents accidental needle sticks. When the needle has an open notch, the blood between the distal opening and the open notch is not held by capillary action and can drop from the needle.

### SUMMARY

The various embodiments of a needle safety assembly have several features, no single one of which is solely responsible for their desirable attributes. Without limiting the scope of the present embodiments as set forth in the claims that follow, their more prominent features now will be discussed briefly.

Aspects of the present disclosure include a catheter device that includes a needle having a needle tip and a wall, a notch formed through the wall of the needle proximal of the needle tip, a catheter hub with a catheter tube having the needle located therein, and a biocompatible seal can be disposed over the notch and/or located inside the catheter tube. A distal portion of the catheter tube can form a seal with the needle.

The biocompatible seal, which can embody a transparent seal window, of the notch allows a quicker visualization of the blood flashback by viewing through the catheter tube and the window defined by the notch with the transparent seal without compromising a secondary flashback. To enhance blood visualization through the transparent window, the biocompatible seal could be made of a magnifying material, such as being structured with zooming or magnifying effect, luminous material, and/or chromogenic material.

Although the term transparent is used herein, it is understood that a semi-opaque material falls within the scope of the term provided blood flow or the presence of blood can be viewed through the window formed around the notch.

The seal of the notch also prohibits blood from dropping out of the notch after the needle has been removed from the catheter tube by maintaining a capillary action to keep the blood in the needle.

A valve can be located in an interior cavity of the catheter hub. An activator can also be located in the interior cavity of the catheter hub proximal of the valve to press open the valve.

A needle shield to capture and cover the needle tip when the needle is retracted proximally out the catheter hub can be located in the interior cavity of the catheter hub.

The needle shield can also be located in an intermediate hub proximal of the catheter hub.

The biocompatible seal can engage and pull the needle shield out of the catheter hub. The biocompatible material can be clear and visible through the catheter tube.

Another aspect of the present disclosure includes a method for detecting blood flashback. The method can include providing a needle having a needle tip, a wall defining a needle lumen, a notch formed through the wall of the needle, and a biocompatible material disposed over the notch, inserting the sharp distal end of the needle into a vein such that the notch is visible outside of the vein, and observing the notch for the flow of blood flowing within the needle lumen.

A still further aspect, such as an alternative aspect, of the present disclosure is a method of manufacturing a needle assembly comprising: providing a needle hub with a needle comprising a needle tip, a wall defining a needle lumen, a notch formed through the wall of the needle; sealing the notch at least in part with a non-metallic biocompatible material having a thickness, the non-metallic biocompatible material being transparent or semi-transparent for viewing through the thickness; and observing the needle lumen through the non-metallic biocompatible material.

The method can further include inserting the needle through a catheter hub and a catheter tube and extending the needle tip distally of a distal end of the catheter tube.

The method can further include retracting the needle proximally to cover the needle tip with a needle shield.

The needle shield can be supported by a valve opener or an activator located in the catheter hub. The needle shield can also be supported in a third hub located proximally of the catheter hub.

The biocompatible material can engage and pull the needle shield out of the catheter hub, can be bonded to the needle, and can be formed by placing an insert inside the needle and spraying the biocompatible material over the notch.
The method can further comprise placing a needle shield in line with the needle to cover the needle tip.

The method wherein the needle shield can be spring loaded.

Yet another aspect of the present disclosure includes a needle device that includes a needle hub, a needle extending from the needle hub and comprising a needle tip, a wall defining a needle lumen, and a notch formed through the wall of the needle at a proximal location of the needle tip, and a biocompatible material disposed over the notch, wherein blood flowing through the needle is visible through the biocompatible material. The biocompatible material can engage with a needle guard or shield to remove the needle guard or shield from an interior cavity of a catheter hub.

### BRIEF DESCRIPTION OF THE DRAWINGS

These and other features and advantages of the present device, system, and method will become appreciated as the same becomes better understood with reference to the specification, claims and appended drawings wherein:

FIG. 1 is a schematic side view of a safety IV catheter assembly provided in accordance with aspects of the present disclosure;

FIG. 2 shows an embodiment of a sealed needle notch in a top view;

FIG. 3 shows an embodiment of an indwelling needle assembly;

FIG. 4 shows an embodiment of a safety catheter assembly; and

FIG. 5 shows an embodiment of a syringe.

FIG. 6 shows an embodiment of a safety catheter assembly with a safety stop.

FIG. 7 shows an embodiment of a catheter assembly having wings with an introducer needle assembly

### DETAILED DESCRIPTION

The detailed description set forth below in connection with the appended drawings is intended as a description of the presently preferred embodiments of needle assemblies provided in accordance with aspects of the present devices, systems, and methods and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

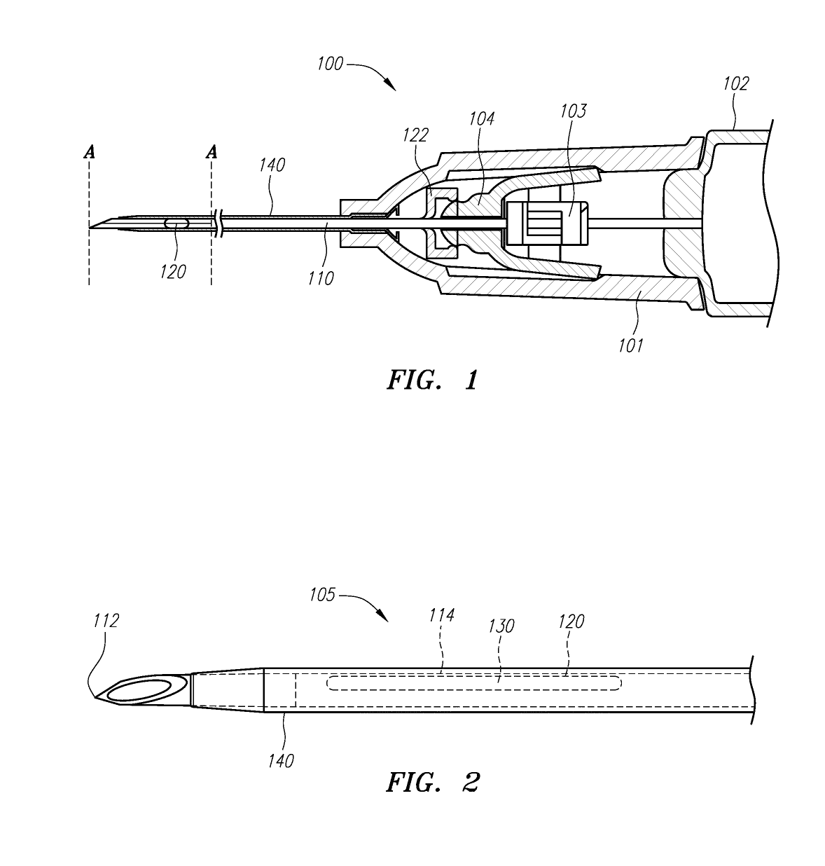

FIG. 1 shows a catheter device or assembly 100 provided in accordance with aspects of the present disclosure. The catheter device 100 includes a catheter hub 101, a needle hub 102, and a needle 110 projecting through a flexible tube or catheter tube 140. A needle tip of the needle extends out the distal end opening of the catheter tube in a ready position. The catheter device 100 can further include a needle shield 103 and an activator 104 for opening a valve 122. The needle 110 can include a change in profile 113, such as a crimp or a bulge, incorporated near the needle tip 112.

The catheter tube 140 is connected to the distal end of the catheter hub 101. The catheter hub is generally conically shaped and defines a hollow interior cavity. The needle 110 is connected to the needle hub 102 and extends from a distal end of the needle hub and passes through the interior of the catheter hub 101 and into the catheter tube 140. The needle tip of the needle extends through the catheter tube 140. The distal portion of the catheter tube 140 can be tapered inward or have an opening that has a size smaller than an outer diameter of the needle 110. Thus the opening of the catheter tube forms a seal with the needle 110 preventing fluid from entering the catheter tube 140 when the needle tip pierces the skin of a patient. Retraction of the needle tip in a proximal direction will allow blood to flow into the space between the needle and the interior of the catheter tube, known as secondary blood flashback.

The valve 122 is housed within the interior cavity of the catheter hub 16 and penetrated by the needle 110 in the ready position shown. The valve 122 can include one or more slits forming flaps (not shown) through which the needle 110 extends. The valve 122 forms a seal around the needle 110. When the needle 110 is withdrawn from the catheter hub 101 after placement of the flexible tube 14 in the patient's vasculature, the slit closes such that the valve 122 seals upon itself thereby restricting or limiting flow across the valve. The valve 122 thus restricts back bleed through the catheter hub 101. The valve 122 is constructed of a material that forms a seal at the interface with the needle 110 and reseals upon itself after the needle 110 is withdrawn. For example, and without limitation, the valve 122 can comprise silicone, silicone rubber, polypropylene, or other suitable materials.

The activator 104 is provided to press against the valve 122 when moved by a medical implement to open the valve 122 to allow fluid or solution to pass through the valve. The activator 104 can be disposed in the interior of the catheter hub 101 for opening the valve 122 by having a passage for receiving the needle in the ready position. After the needle 110 and needle hub 102 are removed, a male medical implement, such as a Luer tip (not shown), a male luer connector, such as used in connection with an IV line, a luer access connector, a syringe tip, or a vent plug, is inserted to push the activator into the seal 122 to open the seal. For example, the activator 104 can be advanced distally by a syringe tip, which presses against the proximal end of the activator 104 to push a nose section of the activator 104 distally forward into the valve 122 to open the one or more slits. In one example, the activator 104 has a wedge shaped nose section to press open the valve 122 and an extension or leg extending in a proximal direction from the nose section to be pushed against by a male medical implement. Although a single extension or leg is usable to push the activator 104, two or more extensions are preferred. The extension can be one or more separate sections that can be pressed against by a male medical implement to advance the activator 104 against the valve 122. The activator 104 has an opening defined through a center of the nose section of the activator 104 for the needle 110 to pass therethrough. Examples of the activator 104 can be found as the valve opener presented in U.S. patent application Ser. No. 14/062,081, the portion of which is incorporated herein by reference.

In one embodiment, the needle shield 103 is located in the interior cavity of the catheter hub 101 and has the needle 110 passing therethrough. The needle shield 103 is configured to cover the tip of the needle 110 after the needle 110 is withdrawn from the catheter hub 101 to prevent needle stick injuries. Examples of a needle shield 103 can be found as the needle guard presented in U.S. Pat. No. 8,827,965 and as the needle safety element presented in U.S. patent application Ser. No. 13/257,572, the portions of which are expressly incorporated herein by reference. The needle shield 103 can also be supported by the activator 104. For example, the activator can have supports supporting the needle shield 103. In an example, the activator 104 has a structure that allows the two arms of the needle shield 103 to rest thereon so that the two distal ends of the needle shield 103 are spaced from the needle 110 in a ready position. The interior cavity of the catheter hub 101 can also support the needle shield 103. In yet another example, the needle shield 103 can be supported or housed in an intermediate hub between the catheter hub 101 and the needle hub 102. The intermediate hub can be removably coupled with the catheter hub 101 and may be referred to as a third hub or a needle shield housing.

FIG. 2 shows a detail of section AA of FIG. 1. Specifically, FIG. 2 shows a notch 120 typically provided on a side of the needle for blood to flow out from the needle lumen through the notch 120 and into the space between the needle and the catheter tube.

In the present embodiment, the notch 120 is sealed with a transparent biocompatible material 130, such as a transparent window cover, thus preventing fluid from passing through the notch 120. In other words, fluid flowing through the needle is confined within the needle when the transparent window cover is incorporated. For example, the perimeter 114 of the notch 120 is sealed with the transparent biocompatible material 130, thus forming the transparent window cover, so that a visual confirmation can be made of a proper venipuncture by viewing through the notch without having to rely on fluid flowing through the notch 120. Due to the presence of the transparent window cover, blood cannot flow through the annular space between the needle 110 and the catheter tube 140. Thus, while blood can be viewed through the catheter tube 140 and the transparent window cover located at the notch 120, no actual blood will flow into the space between the catheter tube and the needle through the notch during the initial stage of catheterization. Blood can flow into the annular space after the needle 110 retracts proximally of the catheter tip, which is a secondary flashback. With secondary flashback, the presence of the catheter tube and the needle tip in the vein are confirmed. In some examples, after blood is viewed through the transparent window for primary flashback, the catheter tube is advanced while the needle is retracted in the proximal direction for blood to flow through the annular space between the needle and the catheter tube, i.e., secondary flashback. However, blood flowing through this space does not flow through the notch because of the transparent window cover or biocompatible seal.

The needle notch 120 may be of any size or shape, such as circular, oval, polygonal, or irregular, but will generally include four edges, including two longitudinal edges and two transverse edges, forming a generally square or rectangular opening. The needle notch 120, and, correspondingly, the sealed needle notch 120, may have rounded or squared corners. The notch 120 can have various sizes while still providing adequate rigidity for performing the medical procedure. Further, as the biocompatible material 130 can provide extra strength to the needle 110, the needle 110 is less likely to bend or kink when performing a venipuncture while still providing visual feedback. Further, the notch 120 should be positioned far enough proximally on the needle tip 112 such that at least a portion of the notch 120 is not inserted into the patient, leaving that portion of the notch 120 visible to the user. This leaves a range of placement for the notch 120 anywhere from just proximal of the needle tip 112 and preferably proximal of a change in profile, such as a crimp or a bulge.

The biocompatible material 130 that forms the transparent window cover can be a glass fused to the notch 120. The biocompatible material 130 can also be a clear polymer such as an ultrathin walled tubing of clear polyamide or a (COC) cyclo-olefin-copolymer. The biocompatible material 130 can also have a magnifying effect, a luminous effect, or both. The magnifying effect can be accomplished by forming the shape of the biocompatible material 130 in the notch 120 with an enlarging function, such as by utilizing a convex or a bi-convex structure through the material to enlarge the viewing image subjacent the structure. For example, with a small gauge needle, the window cover with a magnifying surface facilitates visual feedback through the notch of the presence of blood. The magnifying surface is understood to be a surface feature of the seal material that changes the optics when viewing through the window, for example a convex surface or a bi-convex surface. Further, it is possible to include different material combinations for the window cover, similar to a target having two or more rings with the inner ring, such as the center target, or one of the outer rings made from a non-metallic biocompatible material. Thus, when the notch is sealed with a non-metallic biocompatible material having a thickness, the notch may be sealed, at least in part, with a non-metallic bio-compatible material in combination with other material types, such as other non-metallic materials or metallic materials. Thus, if the window cover is made from two or more different materials, one part of the window cover can be made from a non-metallic bio-compatible material. The non-metallic biocompatible material being transparent or semi-transparent for viewing through the thickness of the window cover to view into the needle lumen.

The biocompatible material 130 can also provide a luminous effect, which reacts with blood or biomaterial to indicate the presence thereof. In an example, the biocompatible material 130 is made from a member of the chromogenic polymers which can change its color or optical properties based on the applied stimulus. The applied stimulus could include temperature, pressure, voltage, ion concentration, biochemical reaction, or light to highlight the presence of blood or other fluid. Therefore, the shape and/or choice of material for the biocompatible material can further highlight the presence of blood or fluid flowing through past the notch 120 and biocompatible material 130. In one example, the transparent window cover is formed and then attached to the needle 110 such as by adhesive or bonding. In another example, an insert, similar to a stylet, is placed inside the needle 110 and the biocompatible material 130 is sprayed on and over the notch 120 and subsequently cured to form a window cover around the notch. The window cover 130 can also be pressed fit into the notch and held thereto by interference. With a sealed notch 120, the user may observe the flow of blood through the catheter tube 140 and needle 110 without any blood spilling out the notch and into the annular space between the needle and the catheter tube.

In another example, the biocompatible material 130 can act as an enlargement, crimp, or change in profile on the needle to engage a proximal opening of the needle shield 103 to pull the needle shield 103 when the needle 110 is retracted proximally out of the catheter hub 101. For example, instead of a separate change in profile, the transparent window cover may incorporate a bump so that when assembled to the notch 120, the bump projects outwardly from the surface of the transparent window cover to engage a proximal wall on the needle shield. The biocompatible material 130 can instead be smooth or substantially flushed with the needle outside surface. In any configuration, the biocompatible material does not completely seal the lumen of the needle, such as the bore of the needle, or prevent fluid from passing through the needle lumen.

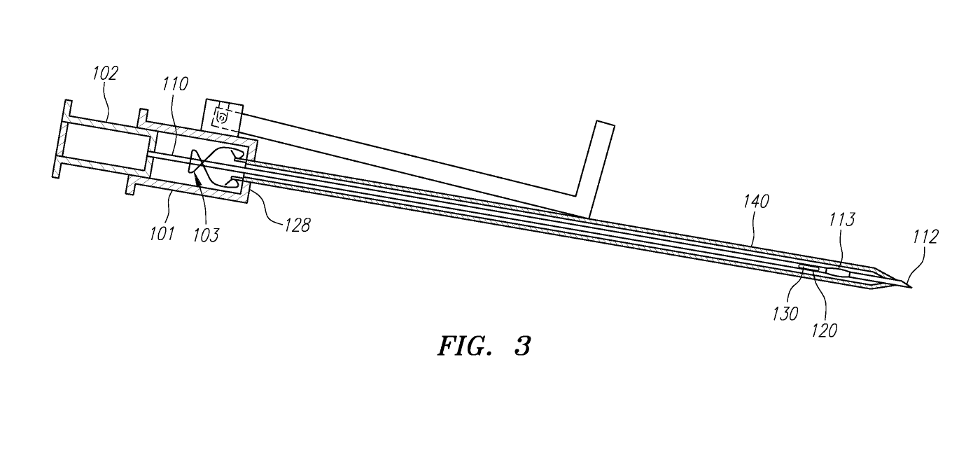

Referring to FIG. 3, an example of one embodiment of an indwelling needle assembly 160 using a combination notch and transparent window cover of FIG. 2 is shown. The indwelling needle assembly 160 can include a needle hub 102 having a needle 110 extending distally through a second hub 101 and a flexible tube 140. A needle shield 103 is located in the second hub 101. The needle shield can be attached to a support located in the interior cavity of the second hub 101 so that the two distal ends of the needle shield are spaced from the needle in a ready position. The needle 110 has a needle tip 112 and a notch 120 proximal to the needle tip 112. A biocompatible material 130 is sealed along a perimeter 114 of the notch 120. Blood flow through the needle can be visible through the biocompatible material for a user to view whether blood is present in the lumen of the needle 110.

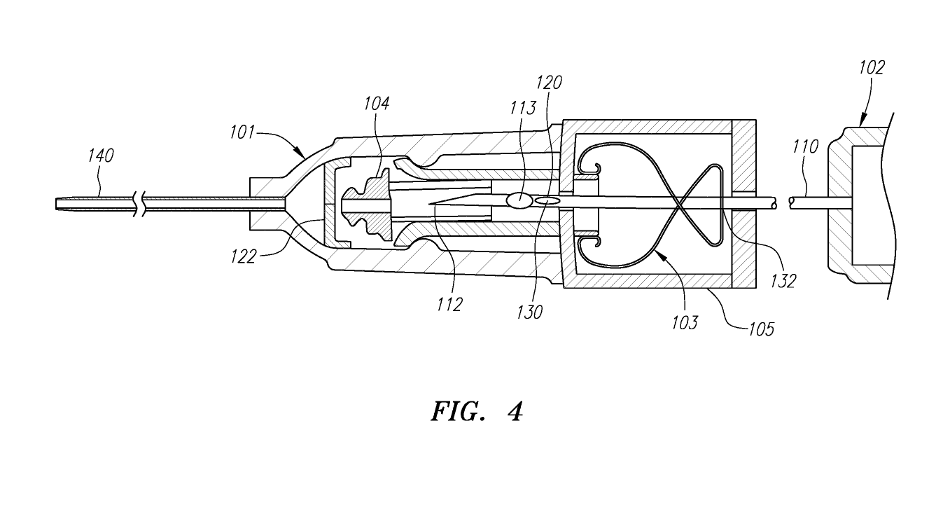

Another example of how the needle 110 with the transparent window cover can be applied is shown with reference FIG. 4. Referring to FIG. 4, one embodiment of a safety catheter assembly 162 is shown similar to the catheter device of FIG. 1, except the needle safety shield 103, which comprises a biasing or resilient member, such as a resilient arm, is completely outside or substantially outside of the catheter hub. As shown, an intermediate hub 105 or needle shield hub is located, at least in part, between the catheter hub 101 and the needle hub 102. The needle 110 also has a change in profile 113 and a notch 120 sealed by biocompatible material 130. A needle shield 103 is located on or in the intermediate hub 105. The intermediate hub 105 can be enclosed as illustrated or can have a single wall. The needle shield 103 can be supported by the intermediate hub or slidably attached to the needle 110 and simply housed in the intermediate hub 105. Alternatively, the needle can incorporate a change in profile directly with the transparent window cover, such as a projection, for engaging an opening on the proximal wall of the needle shield.

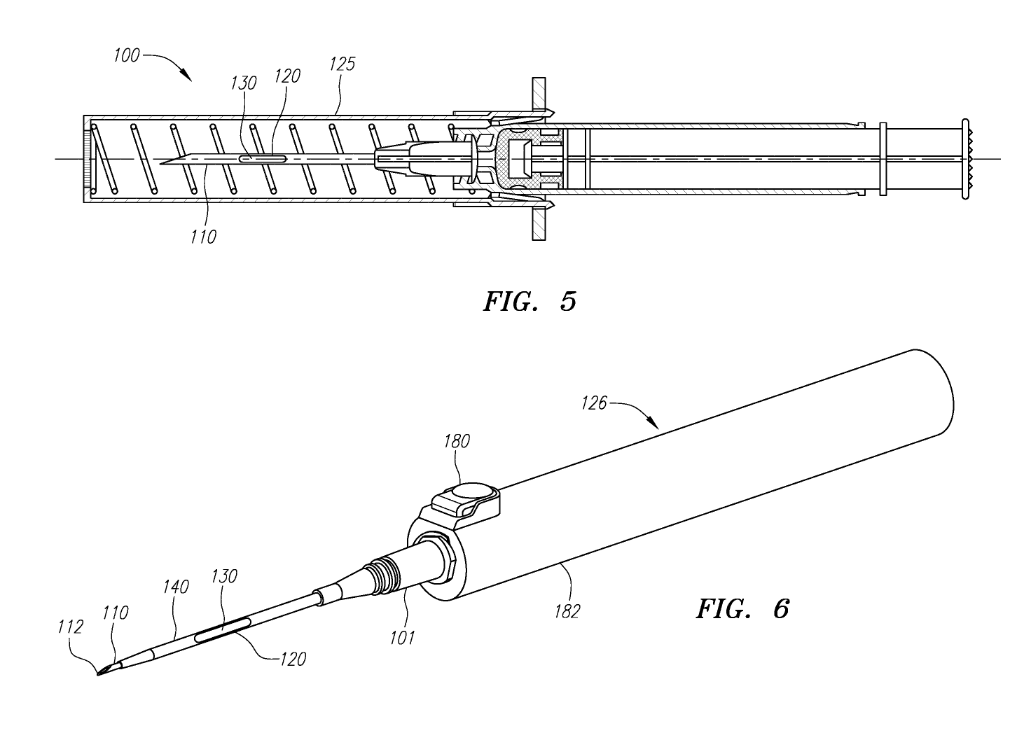

Referring to FIG. 5, a syringe assembly comprising a needle 110 having a notch 120 sealed by a biocompatible material 130 is shown. When the needle is inserted into the patient, fluid flow can be monitored through the biocompatible material 130 in the notch 120. When the injection is complete, a shield assembly 125 is provided that projects over the needle, or the needle is recessed within the shield, thereby preventing accidental needle pricks.

Referring to FIG. 6, a safety catheter assembly 126 having a spring loaded needle carrier that is releasable with or by a safety push button 180 is shown. The needle carrier is attached to a needle 110 comprising a notch 120 sealed by or with a biocompatible material 130 just proximal of the needle tip 112, similar to needles discussed elsewhere herein. The needle 110 projects through a catheter tube 140 of the catheter hub 101 and the needle extends out a distal end of the catheter tube. After the needle 110 and catheter tube 140 are inserted into patient, blood flow can be monitored through the transparent biocompatible material 130 to indicate a successful venipuncture. This allows for inspection of flood flow at an earlier point in time during the procedure than typical secondary blood flashback. Once insertion has been successful, the needle and needle assembly can be removed from the catheter tube by activating the button 180, which releases a spring that then expels the needle carrier and needle into the elongated housing 182 while leaving the catheter tube in place, with the patient.

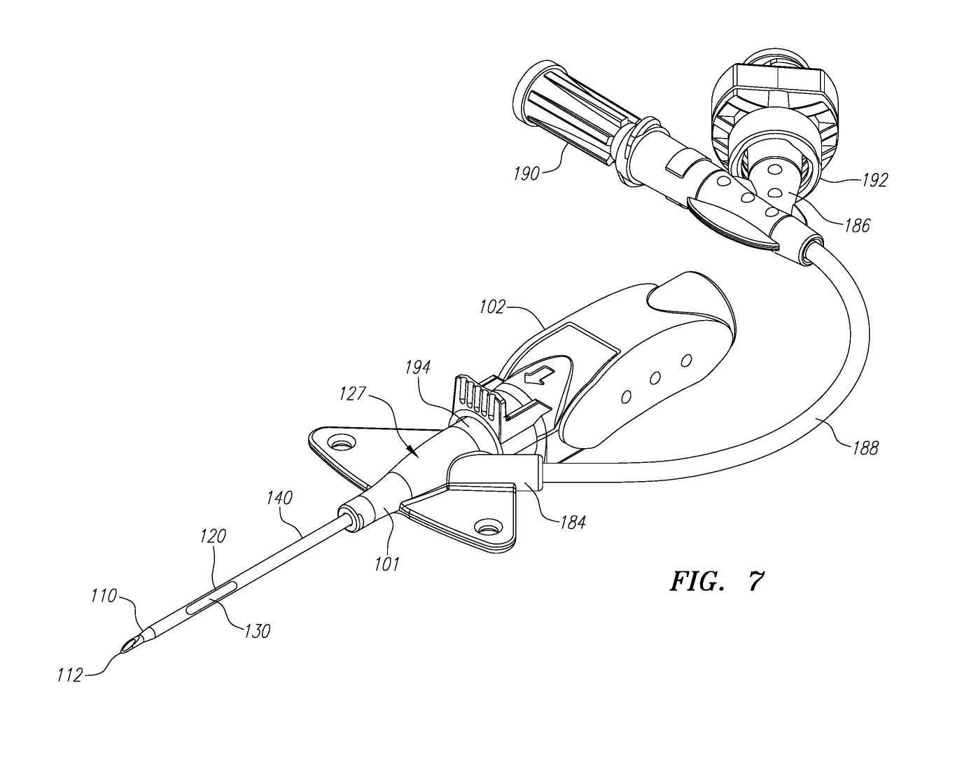

FIG. 7 shows an embodiment of a catheter assembly 127 comprising a catheter hub 101 and a catheter tube 140, a needle hub 102 having a needle 110 extending through the catheter tube 140, a side fluid port 184, and a fluid adaptor 186 attached to the fluid port 184 by a tubing 188 having a lumen for fluid flow between the port and the adaptor. As shown, the fluid adaptor 186 is a Y-site comprising two needleless female Luer connectors 190, 192. The proximal end 194 of the catheter hub 101 is equipped with a septum or a seal and prevents flow thereacross after removal of the needle 110 and the needle hub. The catheter hub 101 is shown with a pair of wings.

A needle tip 112 of the needle 110 extends distally past a distal opening of the catheter tube 140. The needle 110 has a notch 120 and a biocompatible material 130 sealing the notch 120, similar to other needles discussed elsewhere herein. Once inserted into the patient, blood flow can be monitored through the catheter tube and through the biocompatible material 130 at the notch 120. When successful venipuncture has occurred, the needle 110 can be removed from the patient, such as by withdrawing the needle hub 102 in the proximal direction. Fluid can flow through the side port 184 and the fluid adaptor 186.

Methods of manufacturing and method of using needle assemblies, catheter assemblies, and needle devices are also contemplated. For example, aspects of the present disclosure include a method of manufacturing a needle assembly comprising: providing a needle hub with a needle comprising a needle tip, a wall defining a needle lumen, a notch formed through the wall of the needle; sealing the notch, at least in part, with a non-metallic biocompatible material having a thickness, the non-metallic biocompatible material being transparent or semi-transparent for viewing through the thickness; and observing the needle lumen through the non-metallic biocompatible material. For example, following manufacturing, a worker can view through the thickness of the non-metallic biocompatible material to view the inside lumen through the notch to confirm that it is indeed viewable from a position outside the needle.

Although limited embodiments of various needle assemblies with a needle having a sealed needle notch near a needle tip and their components, such as a biocompatible material having a magnifying effect, a luminous effect, and/or made of a chromogenic polymer, have been specifically described and illustrated herein, many modifications and variations will be apparent to those skilled in the art. For example, any over the needle catheter can benefit by using the needle with a sealed notch disclosed herein to provide quicker feedback on whether proper needle placement is made than relying solely on traditional secondary flashback. Furthermore, it is understood and contemplated that features specifically discussed for one needle device having a sealed needle notch embodiment may be adopted for inclusion with another needle device provided the functions are compatible. Accordingly, it is to be understood that the needle devices with sealed needle notch and their components constructed according to principles of the disclosed devices, systems, and methods may be embodied other than as specifically described herein. The disclosure is also defined in the following claims.

1. A catheter device comprising:
a needle comprising a needle tip and a wall;
a notch formed through the wall of the needle proximal of the needle tip;
a catheter hub with a catheter tube having the needle located therein; and
a biocompatible seal disposed over the notch and located inside the catheter tube.

2. The catheter device of claim 1, wherein a distal portion of the catheter tube forms a seal with the needle.

3. The catheter device of claim 2, further comprising a valve located in an interior cavity of the catheter hub.

4. The catheter device of claim 3, further comprising an activator located in the interior cavity of the catheter hub proximal of the valve, wherein the activator is configured to press open the valve.

5. (canceled)

6. The catheter device of any of claim 1, further comprising a needle shield to capture and cover the needle tip when the needle is retracted proximally out the catheter hub.

7. The catheter device of claim 6, wherein the needle shield is located in an interior cavity of the catheter hub.

8. The catheter device of claim 6, wherein the needle shield is located in an intermediate hub proximal of the catheter hub.

9. The catheter device of claim 6, wherein the biocompatible seal engages and pull the needle shield out of the catheter hub.

10. The catheter device of claim 1, wherein the biocompatible seal is transparent and is visible through the catheter tube and is made with a magnifying effect, a luminous effect, or both.

11. The catheter device of claim 1, wherein the biocompatible seal is made from polymer of a class of chromogenic polymers to highlight the presence of blood or fluid flowing through the needle.

12. A method of manufacturing catheter needle assembly comprising:
providing a needle hub with a needle comprising a needle tip, a wall defining a needle lumen, a notch formed through the wall of the needle; and
sealing the notch, at least in part, with a non-metallic biocompatible material having a thickness, the non-metallic biocompatible material being transparent or semi-transparent for viewing through the thickness.

13. The method of claim 12, further comprising inserting the needle through a catheter hub and a catheter tube and extending the needle tip distally of a distal end of the catheter tube.

14. The method of claim 13, further comprising placing a needle shield in line with the needle to cover the needle tip.

15. The method of claim 14, wherein the needle shield is supported by a valve opener located in the catheter hub.

16. The method of claim 14, wherein the needle shield is supported in a third hub located proximally of the catheter hub.

17. The method of claim 14, wherein the needle shield is spring loaded.

18. The method of claim 12, wherein the biocompatible material is bonded to the needle.

19. The method of claim 18, wherein the biocompatible material is formed by placing an insert inside the needle and spraying the biocompatible material over the notch.

20. A needle device comprising:
a needle hub;
a needle extending from the needle hub and comprising a needle tip, a wall defining a needle lumen, and a notch formed through the wall of the needle at a proximal location of the needle tip; and
a biocompatible material disposed over the notch, wherein blood flowing through the needle is visible through the biocompatible material.

21. The needle device of claim 20, wherein the biocompatible material engages a needle shield to remove the needle shield from an interior cavity of a catheter hub.
